(19) United States
(12) Patent Application Publication
(10) Pub. No.: US 2025/0107918 A1
(43) Pub. Date: Apr. 3, 2025

(54) APPARATUS FOR INDUCING AND SUSTAINING AN ERECTION

(71) Applicant: David Evans, Lake Wales, FL (US)

(72) Inventor: David Evans, Lake Wales, FL (US)

(21) Appl. No.: 18/902,409

(22) Filed: Sep. 30, 2024

Related U.S. Application Data
(60) Provisional application No. 63/541,794, filed on Sep. 30, 2023.

(51) Int. Cl. A61F 5/41

(52) U.S. Cl. CPC A61F 5/41; A61F 2005/412; A61F 2005/414; A61F 2005/415

(57) ABSTRACT

A system for inducing and sustaining an erection of a penis a vacuum assembly and a ring assembly adapted to encircle the penis. An inflatable ring assembly disposed in an outwardly flared proximal end of the tube. The ring assembly forms a central circular opening adapted for receiving the penis. A rigid annular ring support is disposed within the ring. A fluid pump is selectively coupled to a valve in the ring assembly, and the pump is adapted to introduce the bladder-inflating fluid under pressure. Expansion of the ring assembly causes the inner surface of the ring assembly to expand while being substantially fixed by the ring support against outwardly radial deformation. A compressive force is thus introduced around the surface of the penis for constricting blood flow from the penis sustain the erection.

### CROSS-REFERENCE TO RELATED APPLICATION

This application claims priority to U.S. Provisional Patent Application No. 63/541,794, filed Sep. 30, 2023, the contents of which are incorporated herein by reference in its entirety.

### BACKGROUND

An apparatus using a vacuum tube for inducing and sustaining an erection of a human penis is described and, more particularly, the erection inducing and sustaining apparatus including a vacuum tube and an inflatable constricting ring for the base of the penis. The apparatus may be used to obtain and maintain an erection in the human male penis, such as in the case to treat erectile dysfunction.

Erectile dysfunction (ED) in human males is a physical condition characterized by the inability to attain or sustain an erection of the penis. A pharmaceutical approach to treating ED includes well known oral drugs, such as Viagra and Cialis. However, these drugs have systemic side effects.

Conventional mechanical means of achieving an erection comprise a hollow cylinder or tube defining a chamber and open at one end for receiving the penis. The tube is connected to a vacuum source such as an air pump. A flaccid penis is inserted into the chamber via the open end of the tube, which is then pressed to the abdomen to provide an airtight seal. The user actuates the vacuum pump and evacuates air from within the chamber of the vacuum tube. The negative pressure created in the chamber around the penis draws blood into the penis creating an erection. When the penis is erect, a compressing ring is slipped into position at the base of the penis. The ring compresses the penis at the base thus trapping the engorged blood and maintaining the erection. Typically, the compressing ring is initially installed on the vacuum cylinder adjacent the open end and is slipped off of the tube and onto the erect penis. The vacuum is then released and the vacuum tube is removed with the ring inhibiting blood flow away from the penis.

Compressing rings for use with the vacuum tube have been designed to be inflatable. The inflatable rings are more effective than simple elastic rings given the variation in size of the human penis. However, the inflatable rings can often be too large and cumbersome when inflated. Moreover, the air pressure within the ring is directed throughout the ring as opposed to directly against the penis where it is needed.

For the foregoing reasons, there is a need for an apparatus for inducing and maintaining an erection using a vacuum tube. Once induced, the new apparatus should comprise an inflatable ring for effectively maintaining tumescence and sustaining the erection. Ideally, the pressure of the inflated ring should be directed radially inwardly against the penis. The new apparatus may be used as an effective treatment of erectile dysfunction.

### SUMMARY

A system for inducing and sustaining an erection of a penis is provided. The penile erection system comprises a vacuum assembly including an elongated tube having an open end and defining a chamber adapted for receiving the penis. An air pump is operatively connected to the tube for evacuating air from the chamber for generating a vacuum within the chamber to induce flow of blood into the penis causing an erection of the penis. A closed ring assembly is adapted to encircle the penis. The ring assembly comprises an annular inflatable bladder having an inner surface and an outer surface and defining a hollow interior between the inner surface and the outer surface for containing a fluid. The inner surface of the bladder forms a central circular opening adapted for receiving the penis. A rigid annular ring support is disposed within the bladder, the ring support having a transverse opening through the support ring. A one-way valve is disposed in the transverse opening of the ring support and opens into the bladder. The valve is configured to control the ingress and egress of a bladder-inflating fluid. A fluid pump is selectively coupled to the valve, and the pump is adapted to introduce the bladder-inflating fluid under pressure through valve and into the bladder. The tube has an outwardly flared proximal end forming an internally recessed annular shoulder for nesting the ring assembly within the proximal end of the tube such that the inner surface of the bladder is adjacent an inner edge of the shoulder. Expansion of the bladder causes the inner surface of the bladder to expand while being substantially fixed by the ring support against outwardly radial deformation. A compressive force is thus introduced around the surface of the penis for constricting blood flow from the penis in order to maintain the erection.

In one aspect, the bladder comprises an elastomeric material, which may be a light silicone rubber.

In another aspect, the fluid introduced into the bladder may comprise air or liquid.

In another embodiment, a selective pressure release button may be provided integral with the valve for adjusting pressure in the bladder.

In yet another embodiment, an inflation needle is provided, wherein the one-way valve is constructed to receive the inflation needle in an aperture through the valve for selectively adding fluid and to prevent the escape of fluid upon removal of the needle from said valve.

A method for inducing and sustaining an erection of a penis is also provided. The penile erection method comprises the steps of providing a vacuum assembly including an elongated tube having an open end and defining a chamber adapted for receiving the penis. An air pump is operatively connected to the tube for evacuating air from the chamber for generating a vacuum within the chamber to induce flow of blood into the penis causing an erection of the penis. A closed ring assembly is adapted to encircle the penis. The ring assembly comprises an annular inflatable bladder having an inner surface and an outer surface and defining a hollow interior between the inner surface and the outer surface for containing a fluid. The inner surface of the bladder forms a central circular opening adapted for receiving the penis. A rigid annular ring support is disposed within the bladder, the ring support having a transverse opening through the support ring and a one-way valve disposed in the transverse opening of the ring support opening into the bladder. The valve is configured to control the ingress and egress of a bladder-inflating fluid. A fluid pump is selectively coupled to the valve, the pump adapted to introduce the bladder-inflating fluid under pressure through valve and into the bladder. The ring assembly is mounted inside the proximal end of the vacuum tube so that the central opening is approximately concentrical with the internal diameter of the vacuum chamber. The vacuum tube is positioned over the penis through the open proximal end of the tube such that the open proximal end is adjacent the groin and the penis passes through the opening in the ring assembly when the ring is in a first uninflated condition. The air pump evacuates air from the tube for generating a vacuum within the chamber adequate to induce an erection of the penis. The bladder is then inflated with fluid to a second inflated condition at which the inner surface of the bladder substantially seals against the base of the erect penis to constrict blood flow out of the penis. The vacuum is released and the tube removed leaving the ring assembly engaged at the base of the penis to restrict the egress of blood for maintaining the erection.

### BRIEF DESCRIPTION OF THE DRAWINGS

For a more complete understanding of the apparatus for use in inducing and sustaining an erection, reference should now be had to the embodiments shown in the accompanying drawings and described below. In the drawings:

FIG. 1 is a front perspective view of an embodiment of an apparatus for inducing and sustaining an erection of a penis.

FIG. 2 is an exploded front perspective view of the erection inducing and sustaining apparatus as shown in FIG. 1.

FIG. 3 is a front elevation view of the erection inducing and sustaining apparatus as shown in FIG. 1.

FIG. 4 is a right side elevation view of the erection inducing and sustaining apparatus as shown in FIG. 1.

FIG. 5 is a longitudinal cross-section view of the erection inducing and sustaining apparatus taken along line 5-5 of FIG. 3.

FIG. 6 is an up-close longitudinal cross-section view of a proximal end of the erection inducing and sustaining apparatus taken along line 6-6 of FIG. 4.

FIG. 7 is a front perspective view of an embodiment of a vacuum tube for use with the erection inducing and sustaining apparatus as shown in FIG. 1.

FIG. 8 is a front elevation view of the vacuum tube as shown in FIG. 7.

FIG. 9 is a longitudinal cross-section view of the vacuum tube taken along line 9-9 of FIG. 8.

FIG. 10 is a top perspective view of an embodiment of a ring assembly for use in the erection inducing and sustaining apparatus as shown in FIG. 1.

FIG. 11 is an exploded perspective view of the ring assembly as shown in FIG. 10.

FIGS. 12A and 12B are a top plan view and a longitudinal cross-section view, respectively, of a ring support for use in the ring assembly as shown in FIG. 10.

FIGS. 13A and 13B are a top plan view and a longitudinal cross-section view, respectively, of an outer ring for use in the ring assembly as shown in FIG. 10.

FIGS. 14A-14C are a perspective view, a top plan view and a longitudinal cross-section view, respectively, of an inner ring for use in the ring assembly as shown in FIG. 10.

FIGS. 15A and 15B are side elevation views of embodiments of an air valve and a modified air valve, respectively, for use in the ring assembly as shown in FIG. 10.

FIG. 16 is a perspective view of an embodiment of a pressure release button for use in the ring assembly as shown in FIG. 10.

FIGS. 17A and 17B are a top plan view and a longitudinal cross-section view, respectively, of a button for use with the pressure release button as shown in FIG. 16.

FIG. 18 is an elevation view of a shaft for use with the pressure release button as shown in FIG. 16.

FIGS. 19A and 19B are an elevation view and a longitudinal cross-section view, respectively, of an embodiment of an inflation needle for use in the ring assembly as shown in FIG. 10.

FIGS. 20A-20D are a top perspective view, a top plan view, an exploded top perspective view and an up close exploded perspective view, respectively, of an assembled embodiment of the ring support as shown in FIGS. 12A and 12B and the valve as shown in FIG. 15B for use with the ring assembly as shown in FIG. 10.

FIG. 21 is an up-close longitudinal cross-section view of the assembled ring support and the valve as shown in FIGS. 20A-20D taken along line 21-21 of FIG. 20B.

FIGS. 22A-22C are a top perspective view, a top plan view, and an exploded perspective view, respectively, of an assembled embodiment of the ring support as shown in FIGS. 12A and 12B, the valve as shown in FIG. 15B, and the pressure release button as shown in FIG. 16 for use with the ring assembly as shown in FIG. 10.

FIG. 23 is an up-close longitudinal cross-section view of the assembled ring support, the valve and the pressure release button as shown in FIGS. 22A-22C taken along line 23-23 of FIG. 22B.

FIGS. 24A-24E are a top perspective view, a bottom perspective view, a side elevation view, a top plan view and an exploded perspective view, respectively, of an assembled embodiment of the ring support as shown in FIGS. 12A and 12B, the valve as shown in FIG. 15B, the pressure release button as shown in FIG. 16, and the outer ring as shown in FIGS. 13A and 13B for use with the ring assembly as shown in FIG. 10.

FIG. 25 is an up-close longitudinal cross-section view of the assembled ring support, the valve, the pressure release button, and the outer ring as shown in FIGS. 24A-24E taken along line 25-25 of FIG. 24D.

FIGS. 26A-26C are a top perspective view, a top plan view and an exploded perspective view of the ring support as shown in FIGS. 12A and 12B, the valve as shown in FIG. 15B, the pressure release button as shown in FIG. 16, the outer ring as shown in FIGS. 13A and 13B, and the inflation needle as shown in FIGS. 19A and 19B for use with the ring assembly as shown in FIG. 10.

FIG. 27 is an up-close longitudinal cross-section view of the assembled ring support, the valve, the pressure release button, the outer ring, and the inflation needle as shown in FIGS. 26A-26C taken along line 27-27 of FIG. 26B.

FIGS. 28A-28C are a top perspective view, a top plan view, and an exploded perspective view, respectively, of the ring support as shown in FIGS. 12A and 12B, the valve as shown in FIG. 15B, the pressure release button as shown in FIG. 16, the outer ring as shown in FIGS. 13A and 13B, the inflation needle as shown in FIGS. 19A and 19B, and the inner ring as shown in FIGS. 14A-14C for use with the ring assembly as shown in FIG. 10.

FIG. 29 is an up-close longitudinal cross-section view of the assembled ring support, the valve, the pressure release button, the outer ring, the inflation needle, and the inner ring as shown in FIGS. 28A-28C taken along line 29-29 of FIG. 28B.

### DESCRIPTION

Certain terminology is used herein for convenience only and is not to be taken as a limiting. For example, words such as “upper,” “lower,” “left,” “right,” “horizontal,” “vertical,” “upward,” “downward,” “top” and “bottom” merely describe the configurations shown in the FIGs. Indeed, the components may be oriented in any direction and the terminology, therefore, should be understood as encompassing such variations unless specified otherwise. The words “interior” and “exterior” refer to directions toward and away from, respectively, the geometric center of the core and designated parts thereof. The terminology includes the words specifically mentioned above, derivatives thereof and words of similar import.

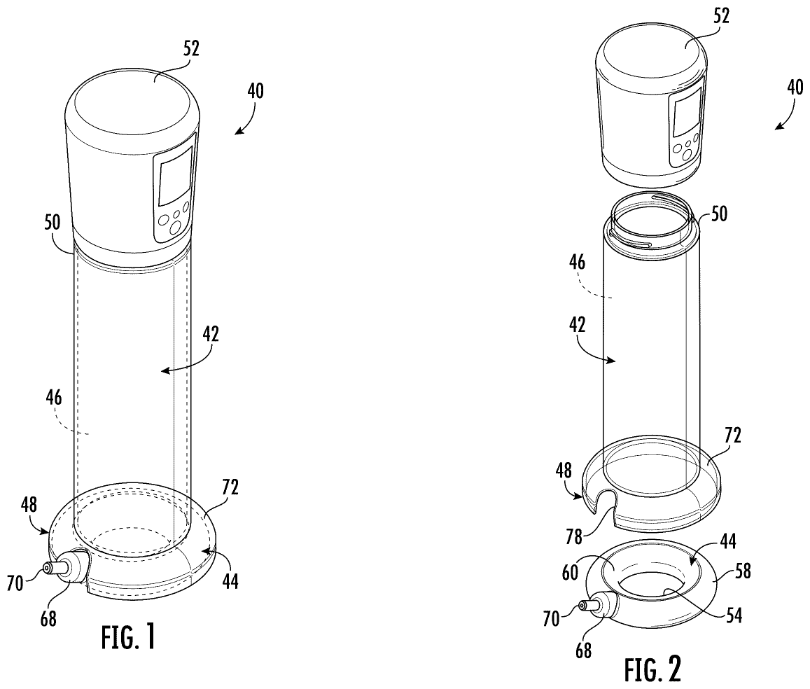

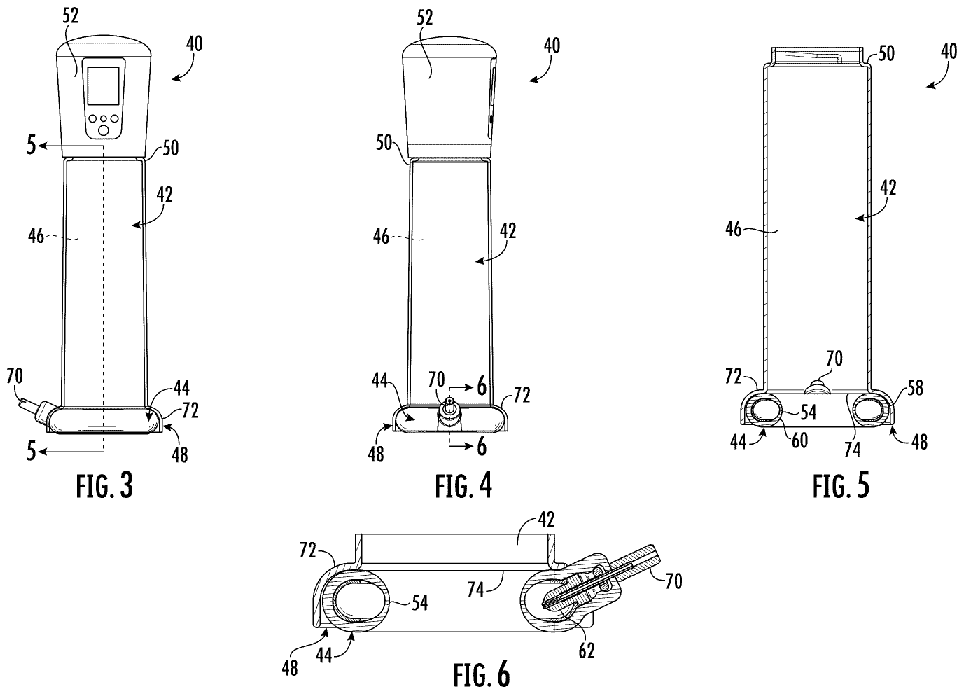

Referring now to the drawings, wherein like reference numbers designate the same or similar elements throughout the several views, an apparatus for inducing and sustaining an erection of the penis is shown in FIGS. 1-5 and generally designated at 40. The apparatus 40 comprises a vacuum tube 42 and a compression ring assembly 44. The vacuum tube 42 is an elongated, generally cylindrical hollow member open at both a proximal end 48 and a distal end 50. The vacuum tube 42 defines an inner chamber 46 between the ends 48, 50. The chamber 46 is configured to receive a human penis for generating an erection of the penis inserted into the vacuum tube 42. The vacuum tube 42 may be formed as a unitary molding of a synthetic plastics material. Preferably the vacuum tube 42 is transparent so that progress can be monitored by the user.

The distal end 50 of the vacuum tube 42 is threaded for operable attachment to a conventional vacuum generating device, such as an air pump 52. The vacuum pump 52 is sealingly mounted by a threaded connection to the distal end 50 of the vacuum tube 42. The vacuum pump 52 is applied to induce tumescence of the penis. The vacuum pump 52 functions to evacuate air from the vacuum tube 42 for generating negative pressure in the chamber 46 resulting in penis expansion and erection. It is understood that the vacuum pump 52 may be a conventional air pump that is electrically-powered or battery-powered.

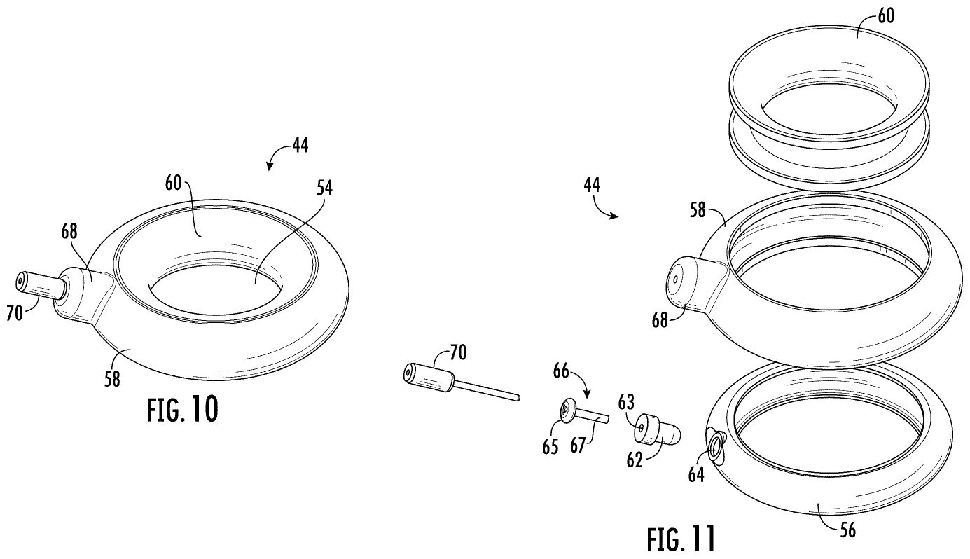

The ring assembly 44 is removably positioned within the proximal end 48 of the vacuum tube 42 (FIGS. 1 and 3-6). Referring to FIGS. 10 and 11, the ring assembly 44 is a generally donut-shaped piece having a circular central opening 54. The ring assembly 44 has an external diameter and an internal diameter. The ring assembly 44 is inflatable having an uninflated condition and an inflated condition rendering the internal selectable by the user. The internal diameter of the opening 54 in the ring assembly 44 in the uninflated condition is configured for passing the penis as the penis is inserted into the chamber 46. For example, the opening 54 in the ring assembly 44 in the uninflated condition may have an inner diameter of about 2¼ inches. It is understood the central opening 54 may be different sizes to accommodate different penis sizes. Each differently sized ring assembly 44 may be matched with a different specifically sized vacuum tube 44 for each specific ring opening size.

The ring assembly comprises a rigid ring support 56, an outer ring 58 and an inner ring 60. As will be described below, the outer ring 58 and the inner ring 60 surround the ring support 56 and define an annular, inflatable bladder 61 along the inner wall of the ring support 56. The ring support 56 accommodates a one-way valve 62 opening into the bladder 61. The valve 62 is configured to control the ingress and egress of a bladder-inflating fluid medium, either air or a liquid. A pump (not shown) is coupled to the valve 62 to introduce the fluid into the bladder 61 via an inflation needle 70. The user may the inflate the bladder 61 by action of the interconnected pump to constrict the erect penis before the vacuum is released. Radially inward expansion of the bladder 61 provides a distributed force around the penis shaft surface at the base of the penis to hold the ring assembly 44 in place on the penis and maintain an erection. The vacuum may be released and the vacuum tube 42 removed and the pump disconnected from the ring assembly 44. The erect penis is then exposed with just the ring assembly 44 in place.

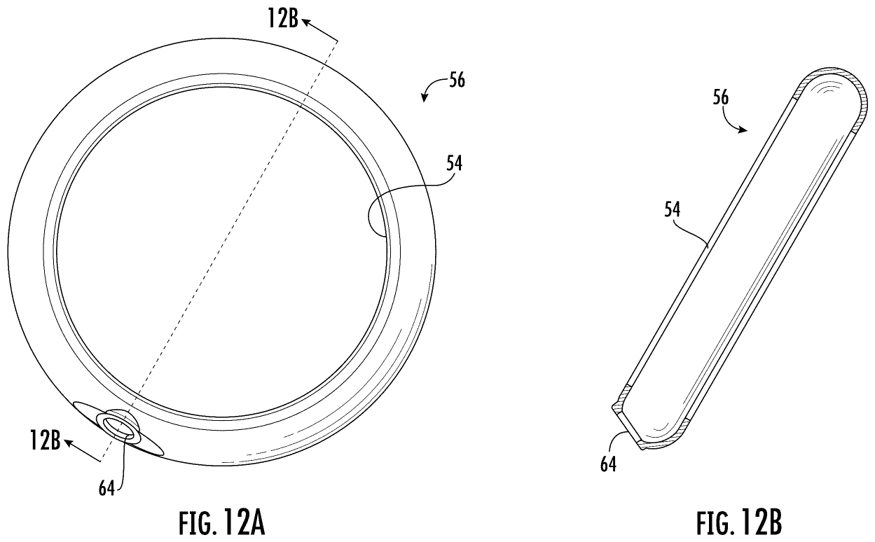

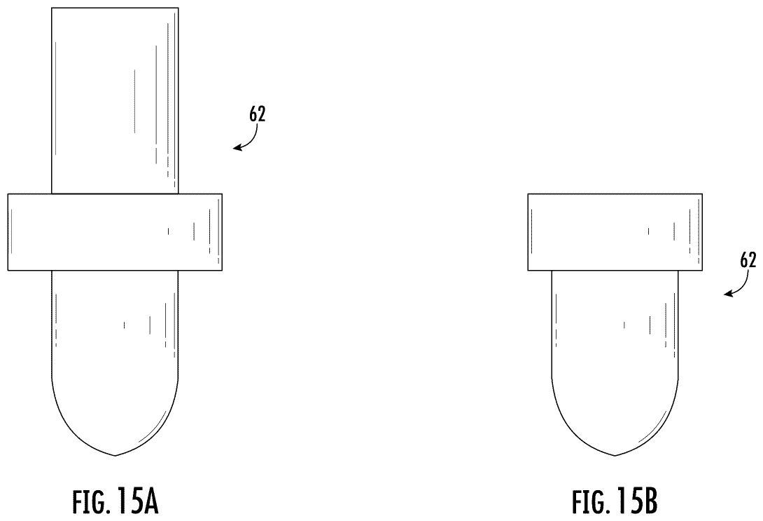

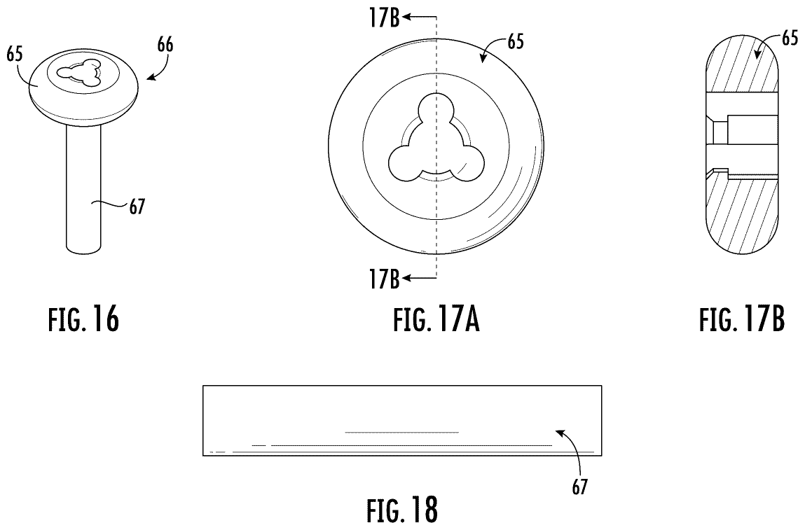

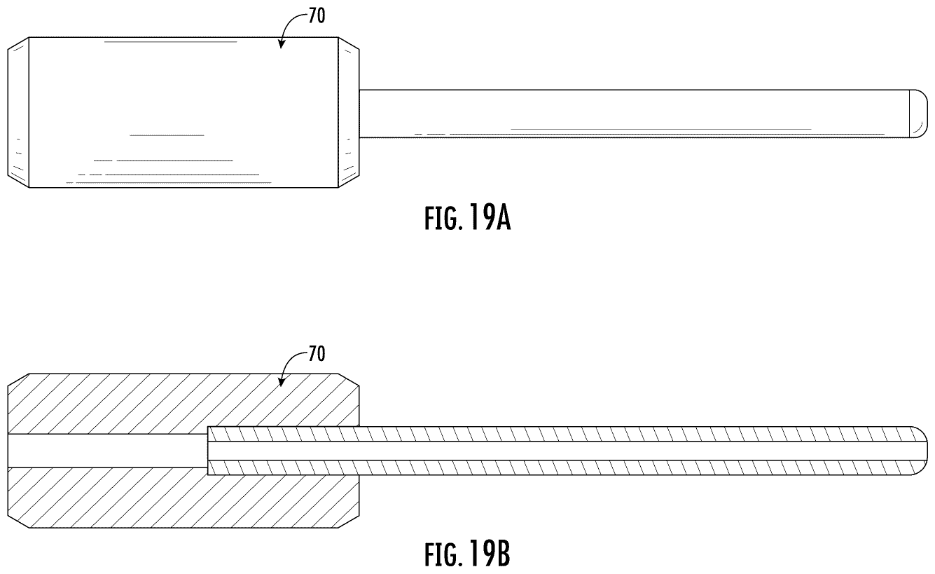

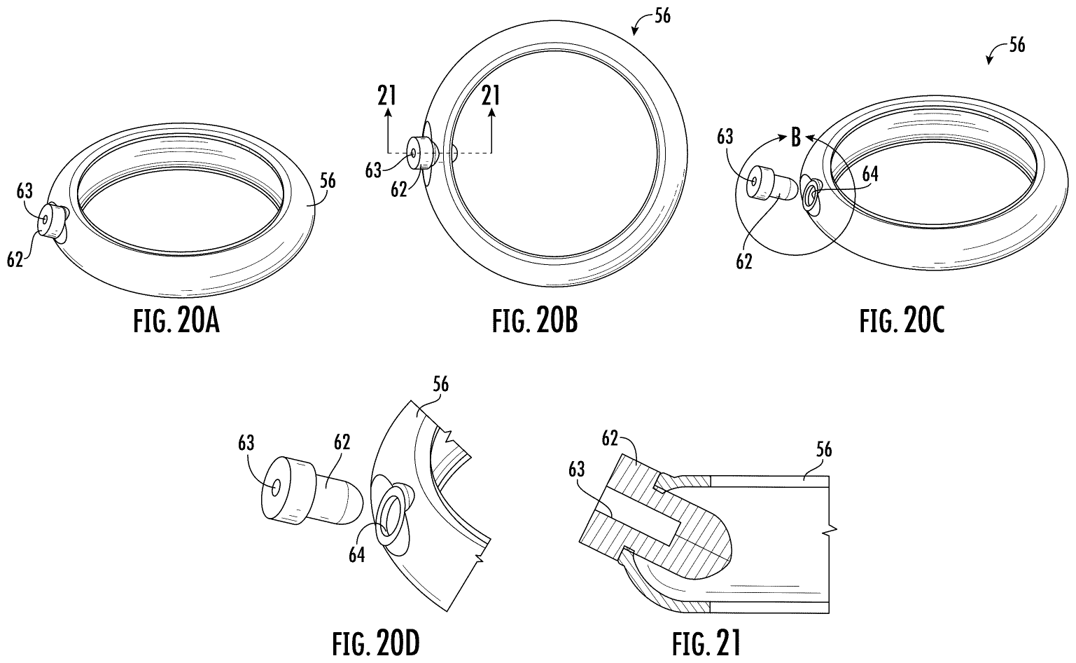

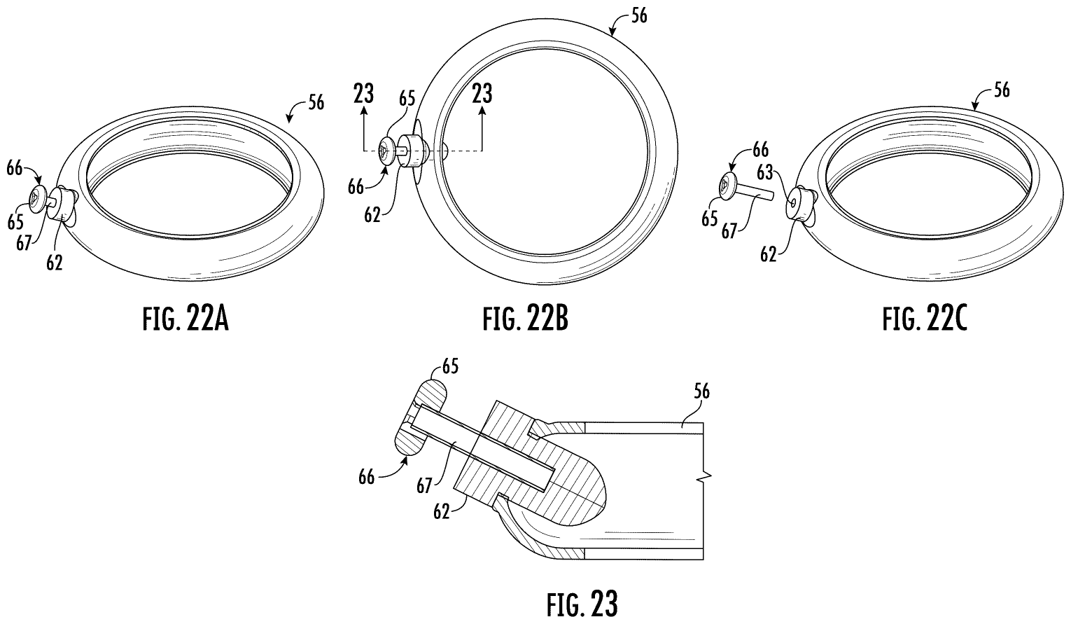

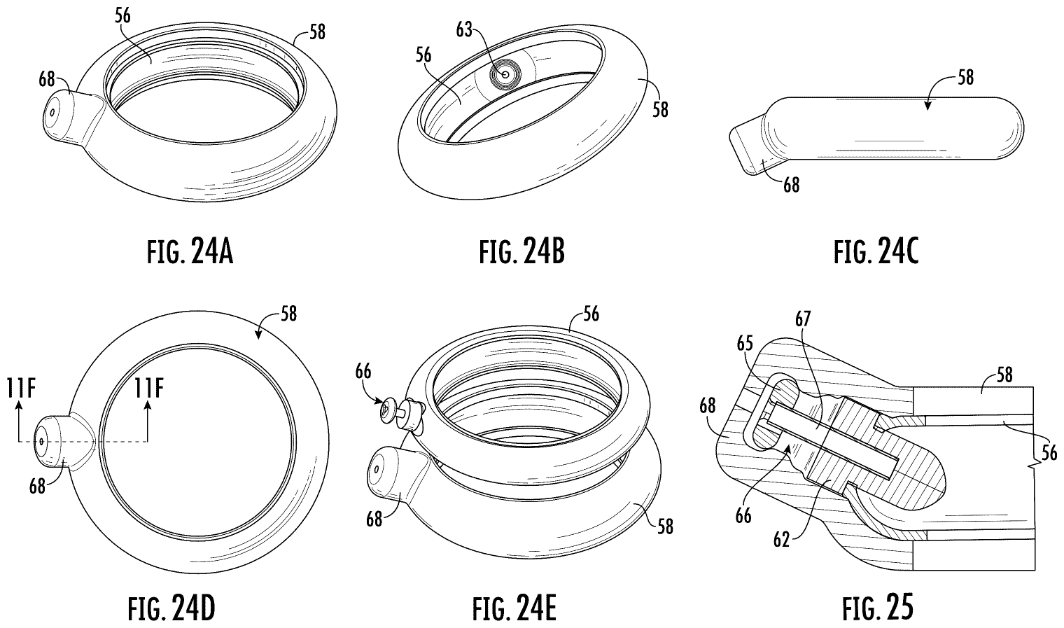

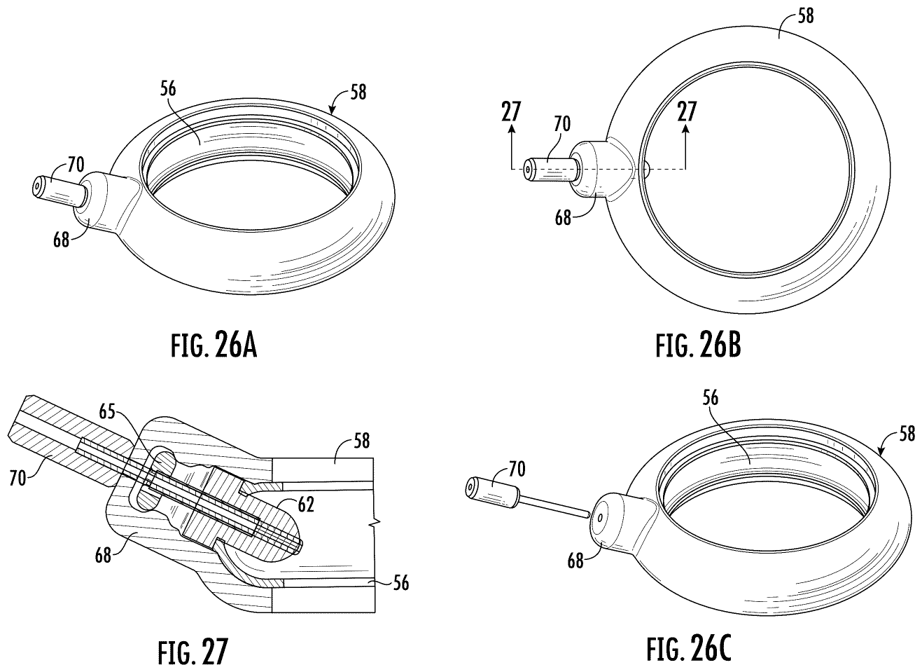

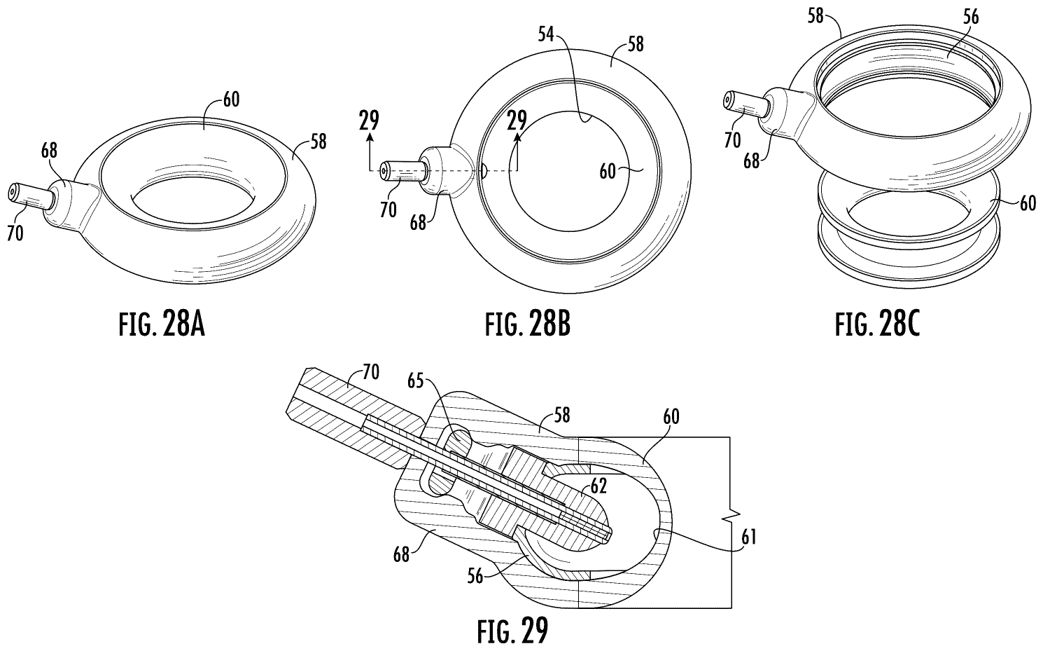

The ring support 56 prevents outward transverse radial expansion of the bladder 61 upon inflation. As seen in FIGS. 12A and 12B, the rigid circular ring support 56 is generally C-shaped in transverse cross-section and has a fixed diameter. Rigidity is provided by the material of the ring support 56, which may be polycarbonate or other suitable relatively rigid material that holds its circular shape and diameter, such as hard rubber. The ring support 56 defines a pass-through transverse opening 64 along its circumference. The opening is sized to sealingly receive the one-way valve 62 and an integral pressure release button 66 (FIGS. 16-18). The valve 62 (FIGS. 15A and 15B) comprises a flexible rubber one-way check valve 62. As seen in FIG. 23, the shaft 67 of the pressure release button 66 is hollow and is in fluid communication with a hole in the button 65 to the atmosphere. The shaft 67 extends only partially through the valve 62 so that the air seal is not broken. The user may push the button 65 inwardly forcing the shaft 67 past the inner end of the valve 62 for allowing fluid pressure within the bladder 61 to escape to the atmosphere.

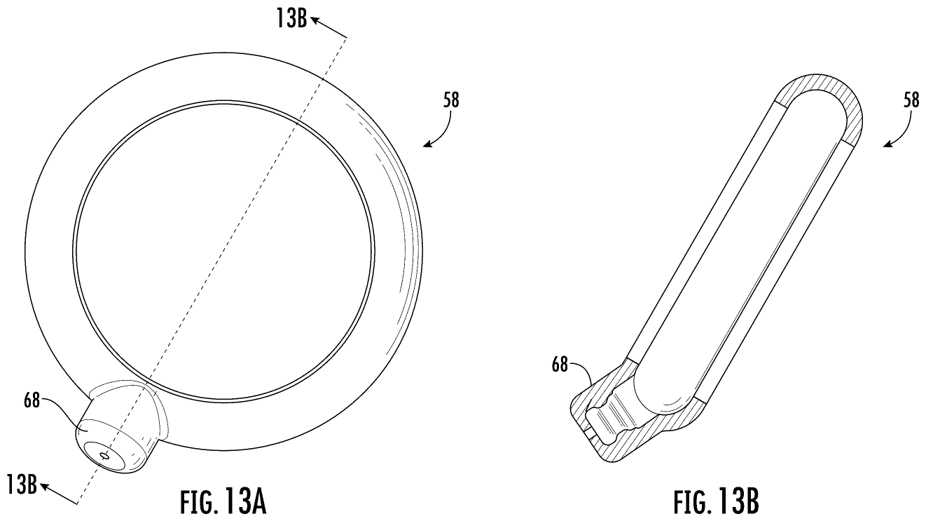

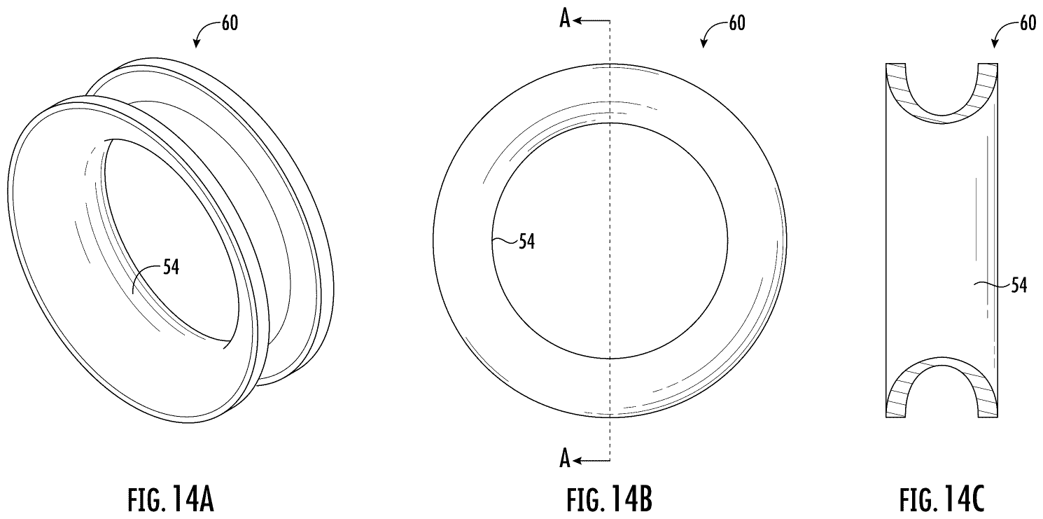

As seen in FIGS. 13A-14C, the outer ring 58 and the inner ring 60 are also circular and generally C-shaped in transverse cross-section. The outer ring 58 and the inner ring 60 are formed from a thin layer of a flexible, elastomeric material, such as a light silicone rubber (LSR) so that they are smooth and pliable. The LSR may have a hardness of from about Shore 30 to about Shore 60, and inner ring may have a different hardness than the outer ring. The outer ring 58 is sized to fit over the outer circumferential surface of the ring support 56 in the manner of an outer sleeve so that the ring assembly 44 does not have rough edges exposed. The outer ring 58 includes a protrusion 68 extending radially outwardly from the body of the outer ring 58. As seen in FIG. 25, the protrusion 68 fits over the valve 62 and the pressure release button 66 in the ring support 56. The protrusion 68 of the outer ring 56 has an opening corresponding to the opening in the button 65 for passing the inflation needle 70 into the valve 62 (FIG. 27). This arrangement functions as an inflation port 76 for the ring assembly 44.

The inner ring 60 is sized to fit along the inner surface of the ring support 56 such that the inner ring 60 is coextensive with the internal circumference of the outer ring 58. As seen in FIG. 29, the inner edges of the rings 58, 60 are adhesively joined to one another to form the airtight, inflatable bladder 61 within the ring assembly 44. It is understood that the embodiment of the two piece bladder 61 shown in the drawings is an example, and a one-piece bladder could also be used. In both embodiments, the bladder should suitably airtight and inflatable within the ring assembly 44. The bladder 61 is inflatable when supplied with compressed fluid via the valve 62. Due to the elasticity of the material, the bladder 61 will expand radially higher air pressure. The expansion is only radially inwardly because outwardly radial expansion is impeded by the rigid ring support 56. As a result, the central opening 54 through the ring assembly 44 becomes smaller. If the penis is in the opening 54, a clamping of the penis will take place for constricting blood flow from the penis.

The valve 62 opens to allow air flow into the bladder 61 when a conventional inflation needle 70 is wedged therein (FIG. 27). In one embodiment, the needle 70 is connectable to a tube for operational communication with the air pump. In one example, the air pump is a handheld pump; however, additional suitable pumps are contemplated as within scope of this invention including, for example, electric or battery powered pumps. The valve 62 closes when the needle 70 is removed. With the needle 70 in place, the user can manually reduce the pressure within the bladder 61 by pushing the needle 70 forcing the pressure release button 66 inwardly. The shaft 67 advances inwardly breaking the seal at the tip of the valve 62, which allows air to escape incrementally for deflating the bladder 61 for either a more comfortable fit, or for deflation of the bladder 61 when penile erection is no longer needed.

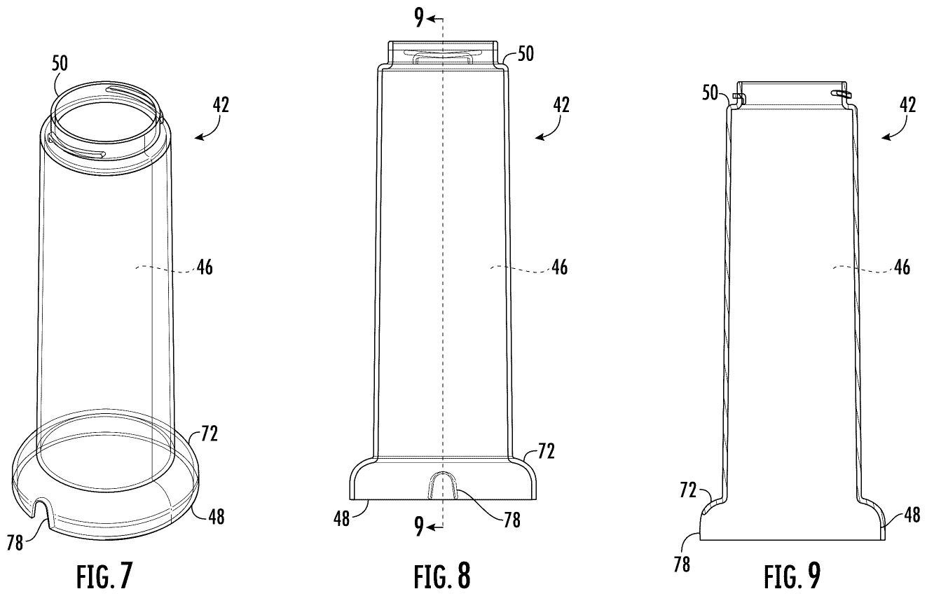

As shown in FIGS. 1-9, the proximal end 48 of the vacuum tube 42 is radially flared for providing a proximal portion 72 of the tube 42 having a larger diameter. The transition of the inner surface of the vacuum tube 42 to the larger diameter defines a continuous, circumferential shoulder 74 adjacent the proximal end of the vacuum tube 42. The shoulder 74 provides a recessed seat internal to the vacuum tube 42 for receiving and supporting the ring assembly 44. The internal diameter of the vacuum tube 42 proximal of the shoulder 74 approximates the external diameter of the compression ring 14 for an interference fit. The internal diameter of the vacuum tube 42 distal of the shoulder 74 and the ring assembly 44 are substantially the same with the bladder 61 deflated. In one embodiment, the internal diameter of the opening 54 in the ring assembly 44 may be slightly less than the internal diameter of the tube 42 distal of the shoulder 74. In either embodiment, the inner surface of the ring assembly 44 is flush or substantially flush with the inner surface of the vacuum tube 42 distal of the proximal end 48 opening. In effect, the ring assembly 44 becomes an extension of the vacuum tube 42 which allows full recruitment of tissues adjacent the proximal end of the tube 41 into the vacuum tube 42, as described hereinbelow. In addition, this arrangement is designed to allow the ring assembly 44 in the inflated condition to separate from the vacuum tube 42 and remain on the penis to serve its function as a constricting ring. Specifically, the inflated ring assembly 44 is freed from the tube 42 when the vacuum is released and the tube removed to seal against the penis to maintain the erection.

The protruding inflation port 76 passes through a slot 78 in an anterior central portion of the proximal end 48 of the vacuum tube 42. The ring assembly 44 may be interconnected in fluid communication with the air pump for selective inflation of the bladder 61 via an air tube (not shown) connect to the inflation needle 70. The air tube is accommodated through the transverse port 76 in the vacuum tube 42. The port 76 ensures the patency of the tube is maintained. The tube may be connected to a manual, handheld, or other air pump without removing the vacuum tube or breaking the airtight seal achieved when the vacuum is initiated to induce the erection. This arrangement also acts to secure the ring assembly 44 in place on the shoulder 74 at the proximal end 48 of the vacuum tube 42. The inflation port 76 is stabilized in the ring assembly 44 so that high pressure does not expel the port extension. The ring assembly 44 is removable once the desired level of bladder 61 inflation is accomplished for sealing against the base of the penis for maintaining an erection.

In use, the user places the penis into the vacuum tube 42 through the opening 54 in the ring assembly 44 at the proximal end 48 of the vacuum tube 42 while the bladder 61 is in an uninflated condition. In one embodiment, the ring assembly 44 may be applied around the base of the penis separately from the vacuum tube. Alternatively, operation of the vacuum pump 52 on the distal end 48 of the vacuum tube 42 draws the flaccid penis of a user into the chamber 46 through the central opening 54 in the ring 44 assembly. In either case, the proximal end 48 of the tube 42 is adjacent the groin around the base of the penis. Operating the air pump 52 draws air from the interior chamber 46 of the vacuum tube 42 which decreases the air pressure in the chamber 46 causing the flaccid penis to be drawn into and expand within the vacuum tube 42. Continued use of the air pump 52 results in the air pressure in the chamber 46 being further reduced and blood being drawn into the penis to achieve an erection of the penis in the chamber 46. The air pump may be able to increase or decrease compression until optimal tumescence is achieved. The vacuum tube 42 is configured for full expansion of the penis.

Negative pressure within the vacuum tube 42 further recruits additional tissue of the perineum, the tissue comprising peri-pubic and upper scrotal tissue, as well as providing outward tension on the suspensory ligament. The additional “recruited” tissue expands into the proximal end 48 of the vacuum tube 42 by suction due to the substantially matching inner diameters of the vacuum tube 42 and the ring assembly 44. The recruited tissue is ultimately compressed proximal of the tissue by the ring assembly 44. The result is an increased functional length of the erect penis induced by the vacuum tube 42.

Once penile erection is achieved, the pump and tube (not shown) are connected to the inflation needle 70 inserted into the valve 62 followed by the pumping of fluid into the bladder 61 to raise pressure in the bladder 61 around the shaft at the base of penis including the recruited tissue. The bladder 61 is inflated and expanded to engage with the penis to constrict outflow of blood from the user's penis before the vacuum is released. Once the blader 61 is inflated, the vacuum may be released and the vacuum tube 42 removed leaving only the ring assembly 44 in place to constrict outflow of blood from the penis and maintain tumescence.

The ring assembly 44 is configured to restrict the expansion of the bladder 61 to only a radially inward direction toward the base of the penis. The bladder 61 is shaped in the form of an oval in transverse cross-section with a greater dimension from the inner circumferential edge to the outer circumferential edge so as to take advantage of Barlow's Formula. This arrangement allows for the compression forces provided by the ring assembly 44 during expansion of the bladder 61 being directed toward the inner circumference augmented by greater thickness from side to side of the band and the bladder 24 and due to the generally oval shape. Moreover, the rigid ring support 56 is spaced radially outwardly of the bladder 61 to further restrict outward expansion of the bladder 61 during inflation while the thinner material of the inner circumference of the bladder 61 also promotes expansion in the radially inner direction. The result is incremental decreasing of the inner diameter of the opening 54 in the ring assembly 44 with minimal widening or ballooning of the bladder 61. The bladder 61 expands inwardly until the bladder seals around the penis for occluding blood flow and maintaining the erection. At this point, the ring assembly 44 is stabilized in place. The vacuum tube 42 is then passively refilled with air thereby relieving the vacuum and returning the chamber 46 to atmospheric pressure. The penis stays rigidly erect due to the engagement of the inflated ring assembly 44. The needle 70 is removed by traction, and the vacuum tube 42 is then disengaged from the ring assembly 44. The fully engaged ring assembly 44 remains in place at the base of the now erect penis providing a controlled erection. The restriction caused by the ring assembly 44 inhibits blood flow from the penis to maintain the erection. Once a session is completed, the user can push the pressure release button 66 provided in the ring assembly 44. This opens the valve 62 for emptying the bladder 61 of pressurized fluid. The ring assembly 44 may then be removed and discarded.

Although an apparatus and method for inducing and sustaining an erection of the penis and method have been shown and described in considerable detail with respect to only a few exemplary embodiments thereof, it should be understood by those skilled in the art that we do not intend to limit the apparatus and method to the embodiments since various modifications, omissions and additions may be made to the disclosed embodiments without materially departing from the novel teachings and advantages, particularly in light of the foregoing teachings. For example, any type of suitable pump may be used to evacuate the vacuum tube or to inflate the bladder in the ring assembly, including a simple inflation bulb operatively connected to the vacuum tube of the ring assembly such that the bulb is operable with one hand to evacuate the tube or pressurize the ring. Accordingly, we intend to cover all such modifications, omission, additions and equivalents as may be included within the spirit and scope of the invention as defined by the following claims. In the claims, means-plus-function clauses are intended to sticker the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

I claim:

1. A system for inducing and sustaining an erection of a penis, the penile erection system comprising:
a vacuum assembly including
an elongated tube having an open end and defining a chamber adapted for receiving the penis, and
an air pump operatively connected to the tube for evacuating air from the chamber for generating a vacuum within the chamber to induce flow of blood into the penis causing an erection of the penis; and
a closed ring assembly adapted to encircle the penis, the ring assembly comprising
an annular inflatable bladder having an inner surface and an outer surface and defining a hollow interior between the inner surface and the outer surface for containing a fluid, the inner surface of the bladder forming central circular opening adapted for receiving the penis,
a rigid annular ring support disposed within the bladder, the ring support having a transverse opening through the support ring,
a one-way valve disposed in the transverse opening of the ring support and opening into the bladder, the valve configured to control the ingress and egress of a bladder-inflating fluid, and
a fluid pump selectively coupled to the valve, the pump adapted to introduce the bladder-inflating fluid under pressure through valve and into the bladder,
wherein the tube has an outwardly flared proximal end forming an internally recessed annular shoulder for nesting the ring assembly within the proximal end of the tube such that the inner surface of the bladder is adjacent an inner edge of the shoulder, and
wherein expansion of the bladder causes the inner surface of the bladder to expand while being substantially fixed by the ring support against outwardly radial deformation for introducing a compressive force around the surface of the penis for constricting blood flow from the penis in order to maintain the erection.

2. The penile erection system as recited in claim 1, wherein the bladder comprises an elastomeric material.

3. The penile erection system as recited in claim 2, wherein the elastomeric material is a light silicone rubber.

4. The penile erection system as recited in claim 1, wherein the fluid introduced into the bladder comprises air.

5. The penile erection system as recited in claim 1, wherein the fluid introduced into the bladder comprises water.

6. The penile erection system as recited in claim 1, further comprising a selective pressure release button integral with the valve for adjusting pressure in the bladder.

7. The penile erection system as recited in claim 1, further comprising an inflation needle, wherein the one-way valve is constructed to receive the inflation needle in an aperture through the valve for selectively adding fluid and to prevent the escape of fluid upon removal of the needle from said valve.

8. A method for inducing and sustaining an erection of a penis, the penile erection method comprising the steps of:
providing a vacuum assembly including an elongated tube having an open end and defining a chamber adapted for receiving the penis, and an air pump operatively connected to the tube for evacuating air from the chamber for generating a vacuum within the chamber to induce flow of blood into the penis causing an erection of the penis;
providing a closed ring assembly adapted to encircle the penis, the ring assembly comprising
an annular inflatable bladder having an inner surface and an outer surface and defining a hollow interior between the inner surface and the outer surface for containing a fluid, the inner surface of the bladder forming central circular opening adapted for receiving the penis,
a rigid annular ring support disposed within the bladder, the ring support having a transverse opening through the support ring,
a one-way valve disposed in the transverse opening of the ring support and opening into the bladder, the valve configured to control the ingress and egress of a bladder-inflating fluid, and
a fluid pump selectively coupled to the valve, the pump adapted to introduce the bladder-inflating fluid under pressure through valve and into the bladder,
positioning the ring assembly inside the proximal end of the vacuum tube so that the central opening is approximately concentrical with the internal diameter of the vacuum chamber;
positioning the vacuum tube over the penis through the open proximal end of the tube such that the open proximal end is adjacent the groin and the penis passes through the opening in the ring assembly when the ring is in a first uninflated condition;
actuating the air pump for evacuating air from the tube for generating a vacuum within the chamber adequate to induce an erection of the penis;
inflating the bladder with fluid to a second inflated condition at which the inner surface of the bladder substantially seals against the base of the erect penis to constrict blood flow out of the penis; and
releasing the vacuum and removing the tube leaving the ring assembly engaged at the base of the penis to restrict the egress of blood for maintaining the erection.
